(19) United States
(12) Patent Application Publication
(10) Pub. No.: US 2025/0107984 A1
(43) Pub. Date: Apr. 3, 2025

(54) Antidandruff Shampoo

(71) Applicant: Henkel AG & Co. KGaA, Duesseldorf (DE)

(72) Inventors: Petra Westphal, Neu Wulmstorf (DE); Thomas Schroeder, Hamburg (DE); Manuela Mette, Schönberg OT Kleinfeld (DE); Soeren Scheele, Hamburg (DE); Jenny Marschall, Marschacht (DE); Dirk Hentrich, Hamburg (DE)

(21) Appl. No.: 18/906,773

(22) Filed: Oct. 4, 2024

Related U.S. Application Data
(63) Continuation of application No. PCT/EP2023/050373, filed on Jan. 10, 2023.

(30) Foreign Application Priority Data
Apr. 7, 2022 (DE) 102022203485.6

(51) Int. Cl. A61K 8/37; A61K 8/34; A61Q 5/00

(52) U.S. Cl. CPC A61K 8/375; A61K 8/345; A61Q 5/006

(57) ABSTRACT

Antidandruff shampoos which are based on a natural active antidandruff ingredient a) and at least one surfactant b) and in which a) are compounds of the following formula (I)

R¹—O—[CH₂—CH(OH)—CH₂—O]ₙ—H (I),
in which R¹ represents a branched C₆-C₁₆ alkyl, alkenyl or alkynyl group and n represents a number of 1-3, are suitable for controlling dandruff.

### FIELD OF THE INVENTION

The present invention relates to an antidandruff shampoo comprising specific glycerol ethers and to the cosmetic use of the antidandruff shampoo for cleaning hair and for the prophylaxis, reduction, elimination and alleviation of dandruff on hair-covered body surfaces. The present invention further relates to a cosmetic method for controlling scalp dandruff using the agents.

Even slight dandruff on the scalp or other hair-covered areas of the body is perceived as a sign of lack of care and hygiene. Dandruff is also usually accompanied by a nagging itch, which can lead to scratching and injury to the affected areas of skin. Skin injuries often form the basis for infections and pathogens.

Dandruff can be promoted by various factors, for example, hereditary predisposition, a tendency toward increased sebum production (seborrhea), hormonal fluctuations, stress, climatic conditions or improper hair care. However, the key contributor to the formation of dandruff is the colonization of the scalp by yeast fungi of the genus Malassezia.

### BACKGROUND OF THE INVENTION

In dandruff control, it has therefore always been an aim to reduce and/or eliminate the colonization of the scalp with Malassezia.

For this purpose, numerous cosmetically acceptable and effective active antidandruff ingredients are known from the prior art, such as piroctone olamine, climbazole, zinc pyrithione or other zinc salts, sulfur and/or selenium sulfide.

The effect of these active antidandruff ingredients is mainly based on their fungicidal properties.

However, previously mentioned conventional active antidandruff ingredients have disadvantages. On the one hand, they can in some cases be irritating to the skin, and, due to their poor solubility in water, they are somewhat complicated to incorporate into conventional cosmetic products. In addition, due to their poor biodegradability, they are under increasing pressure and are subject to increasingly stringent regulations or banned from use in cosmetic products.

However, a reduction in the quantity or even a complete replacement of undesired or problematic substances in cosmetic products can only be carried out if alternatives are available that also have the desired properties for the intended use.

Various plant extracts, for example arnica, birch, burdock root, beard lichen, poplar, stinging nettle, myrtle or hazelwort extracts, are known to be active antidandruff ingredients based on natural substances. However, with regard to their immediate and/or long-lasting effectiveness, these extracts cannot completely replace the classic active antidandruff ingredients mentioned above.

There is therefore still a need for compounds having good efficacy against dandruff that can be obtained from natural sources and can be easily incorporated into cosmetic products without problems.

### BRIEF SUMMARY OF THE INVENTION

The object of the invention is accordingly to provide an antidandruff shampoo that reduces and/or eliminates the colonization of the scalp by yeast fungi of the genus Malassezia and comprises a very high proportion of environmentally friendly active antidandruff ingredients.

In addition, the antidandruff shampoos should ensure gentle cleaning and care of the scalp and hair.

Surprisingly, it has been found that certain medium-chain glycerol ethers that can be obtained from sustainable, renewable plant-based raw materials can be excellently incorporated into cosmetic products containing surfactants and allow effective treatment of dandruff conditions, in particular those associated with the colonization and/or proliferation of yeast fungi of the genus Malassezia.

Antidandruff shampoos based on these specific glycerol ethers can in particular also be used on sensitive scalps and have a mild cleaning, balancing and soothing effect.

### DETAILED DESCRIPTION OF THE INVENTION

The present invention is therefore characterized by the following statements:

1. An antidandruff shampoo, containing in a cosmetic carrier
a) 0.05-5 wt. % of at least one compound of the following formula (I)

R¹—O—[CH₂—CH(OH)—CH₂—O]ₙ—H (I)
where
   R¹ represents a branched C₆-C₁₆ alkyl, alkenyl or alkynyl group
   n represents a number of 1-3, and
b) 0.5-20 wt. % of at least one surfactant,
wherein the quantities specified relate to the total weight of the antidandruff shampoo.

2. The antidandruff shampoo according to statement 1, wherein the total amount of the or each compound according to formula (I) ranges from 0.1-3 wt. % relative to the total weight of the antidandruff shampoo.

3. The antidandruff shampoo according to one of the preceding statements, wherein the total amount of the or each compound according to formula (I) ranges from 0.3-1.5 wt. % relative to the total weight of the antidandruff shampoo.

4. The antidandruff shampoo according to one of the preceding statements, wherein n is the number 1 in the or each compound according to formula (I).

5. The antidandruff shampoo according to one of the preceding statements, wherein in the or each compound according to formula (I) R¹ represents a branched C₆-C₁₂ alkyl, alkenyl or alkynyl group.

6. The antidandruff shampoo according to one of the preceding statements, wherein in the or each compound according to formula (I) R¹ represents a branched C₆-C₁₀ alkyl, alkenyl or alkynyl group.

7. The antidandruff shampoo according to one of the preceding statements, wherein in the or each compound according to formula (I) R¹ represents a branched C₈ alkyl, alkenyl or alkynyl group.

8. The antidandruff shampoo according to one of the preceding statements, wherein the compound according to formula (I) is H₃C—(CH₂)₅—CH(CH₃)—O—CH₂—CH(OH)—CH₂OH (methylheptylglycerin).

9. The antidandruff shampoo according to one of the preceding statements, wherein the at least one surfactant b) is selected from anionic surfactants, amphoteric surfactants, zwitterionic surfactants, non-ionic surfactants, or mixtures thereof.

10. The antidandruff shampoo according to one of the preceding statements, containing
b1) 1-15 wt. % of at least one anionic surfactant,
b2) 0-7.5 wt. % of at least one amphoteric or zwitterionic surfactant,
b3) 0-5 wt. % of at least one non-ionic surfactant,
wherein the quantities specified relate to the total weight of the antidandruff shampoo.

11. The antidandruff shampoo according to one of the preceding statements, additionally containing 0.01-3 wt. % of at least one cationic polymer c), wherein the quantities specified relate to the total weight of the antidandruff shampoo.

12. The antidandruff shampoo according to one of the preceding statements, wherein the total amount of the or each cationic polymer c) ranges from 0.02-2 wt. % relative to the total weight of the antidandruff shampoo.

13. The antidandruff shampoo according to one of the preceding statements, wherein the total amount of the or each cationic polymer c) ranges from 0.025-1 wt. % relative to the total weight of the antidandruff shampoo.

14. The antidandruff shampoo according to one of statements 11 to 13, wherein the or each cationic polymer c) is selected from compounds known under the INCI names Guar Hydroxypropyltrimonium Chloride, Polyquaternium-10, Polyquaternium-6, Polyquaternium-67, Polyquaternium-37, or mixtures thereof.

15. The antidandruff shampoo according to one of the preceding statements, additionally containing 0.01-5 wt. % of at least one hair care substance d) different from c), wherein the quantities specified relate to the total weight of the antidandruff shampoo.

16. The antidandruff shampoo according to statement 15, wherein the total amount of the or each hair care substance d) different from c) ranges from 0.025-3 wt. % relative to the total weight of the antidandruff shampoo.

17. The antidandruff shampoo according to one of statements 15 or 16, wherein the total amount of the or each hair care substance d) different from c) ranges from 0.05-2.5 wt. % relative to the total weight of the antidandruff shampoo.

18. The antidandruff shampoo according to one of statements 15 to 17, wherein the or each hair care substance d) different from c) is selected from glycerol esters, natural oils, butters or waxes, ester oils, ethers, C8-C16 alkanes, or mixtures thereof.

19. The antidandruff shampoo according to one of statements 15 to 18, wherein the or each hair care substance d) different from c) is selected from glycerol monooleate.

20. A cosmetic use of an antidandruff shampoo according to one of statements 1 to 19 for cleaning and caring for human hair, and for the prophylaxis, reduction, elimination and alleviation of dandruff on hair-covered body surfaces.

21. A cosmetic method for controlling scalp dandruff, in which an antidandruff shampoo according to one of statements 1 to 19 is applied to the hair and scalp and rinsed out again after a contact time of 5 seconds to 5 minutes.

As a first substantial constituent, the antidandruff shampoos according to the invention contain at least one compound according to formula (I).

Such biobased glycerol ethers are suitable as preservatives in cosmetic or pharmaceutical compositions and as odor control agents in deodorants (WO 2021/237207 A1).

The antidandruff efficacy of medium-chain glycerol ethers has hitherto not been known.

The antidandruff shampoos according to the invention preferably contain compounds according to formula (I), in which n represents the number 1. Preference is also given to compounds according to formula (I), in which R¹ represents a branched C₆-C₁₂ alkyl, alkenyl or alkynyl group, more preferably a branched C₆-C₁₀ alkyl, alkenyl or alkynyl group and particularly preferably a branched C₈ alkyl, alkenyl or alkynyl group.

A particularly preferred compound according to formula (I) is H₃C—(CH₂)₅—CH(CH₃)—O—CH₂—CH(OH)—CH₂OH (methylheptylglycerin—MHG).

It has been found that even small amounts of compounds according to formula (I) are sufficient to achieve satisfactory antidandruff efficacy.

It is also advantageous that compounds according to formula (I) can be combined with other, even conventional active antidandruff ingredients in a surfactant base, and that the antidandruff efficacy can be boosted, even for very small amounts of other active antidandruff ingredients.

Compound(s) according to formula (I) are preferably used in a quantity range of 0.1-3 wt. % relative to the total weight of the antidandruff shampoo. Particularly preferred are total amounts of one or more compound(s) according to formula (I) of 0.2-2 wt. %, and in particular 0.3-1.5 wt. %.

Very particular preference is given to using methylheptylglycerin (MHG) in the aforementioned amounts in the antidandruff shampoos according to the invention, in particular biobased methylheptylglycerin, which can be obtained from sustainable, renewable plant-based raw materials.

Corresponding products are commercially available, for example from Inolex under the trade name Lexgard® Natural MHG MB.

The antidandruff shampoos according to the invention preferably contain components a) and b) in a cosmetic carrier. In the context of the invention, this is understood to be preferably an aqueous or aqueous-alcoholic carrier.

The cosmetic carrier preferably contains at least 70 wt. %, particularly preferably at least 72.5 wt. % and more particularly preferably at least 75 wt. %, water.

Furthermore, the cosmetic carrier may contain 0.01 to 10 wt. %, preferably 0.05 to 9 wt. %, and in particular 0.10 to 6 wt. %, of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures of said alcohols.

Mild and good foaming properties of antidandruff shampoos according to the invention can be controlled by carefully selecting surfactant amounts and/or surfactant types.

Preferably, antidandruff shampoos according to the invention contain at least one anionic surfactant which is responsible for producing satisfactory foam amounts and foaming properties. To achieve an optimal balance between mildness and foaming properties, a mixture of at least one anionic surfactant and at least one mild co-surfactant is particularly preferred. The mild co-surfactant can preferably be selected from amphoteric and/or zwitterionic and/or non-ionic surfactants.

In a preferred embodiment, antidandruff shampoos according to the invention contain
c1) 1-15 wt. % of at least one anionic surfactant,
c2) 0-7.5 wt. % of at least one amphoteric or zwitterionic surfactant,
c3) 0-5 wt. % of at least one non-ionic surfactant,
wherein the quantities specified relate to the total weight of the hair treatment agent.

Suitable anionic surfactants within the meaning of the present invention are:
linear and branched fatty acids having 8 to 30 C atoms (soaps);
ether carboxylic acids of the formula R—O—(CH₂—CH₂O)_{X}—CH₂—COOH, in which R is a linear or branched, saturated or unsaturated alkyl group having 8 to 30 C atoms, and x=0 or 1 to 16;
acyl sarcosides having 8 to 24 C atoms in the acyl group (sarcosinate surfactants);
acyl taurides having 8 to 24 C atoms in the acyl group (taurate surfactants);
acyl isethionates having 8 to 24 C atoms in the acyl group (isethionate surfactants);
sulfosuccinic acid mono- and/or dialkyl esters having 8 to 24 C atoms in the alkyl group and sulfosuccinic acid monoalkyl polyoxyethyl esters having 8 to 24 C atoms in the alkyl group and 1 to 6 oxyethyl groups (sulfosuccinate surfactants);
alpha-olefin sulfonates having 8 to 24 C atoms (alpha-olefin sulfonate surfactants);
alkyl sulfates and/or alkyl ether sulfate salts of the formula R—(OCH₂—CH₂)ₙ—O—SO₃X in which R preferably represents a straight-chain or branched, saturated or unsaturated alkyl group having 8 to 30 C atoms, x is the number 0 or 1 to 12 and X denotes an alkali, alkaline earth, ammonium or alkanolamine ion;
sulfonates of unsaturated fatty acids having 8 to 24 C atoms and 1 to 6 double bonds,
esters of tartaric acid and citric acid with alcohols, which represent addition products of about 2-15 molecules ethylene oxide and/or propylene oxide to fatty alcohols having 8 to 22 C atoms; and/or
alkyl and/or alkenyl ether phosphates of the formula

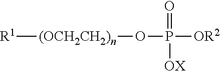

in which R¹ preferably represents an aliphatic hydrocarbon functional group having 8 to 30 carbon atoms, R² represents hydrogen, a functional group (CH₂CH₂O)ₙR¹ or X, n represents numbers from 0 to 10 and X represents hydrogen, an alkali or alkaline earth metal or the group —NR³R⁴R⁵R⁶, where R³ to R⁶ represent, independently of one another, a C¹ to C⁴ hydrocarbon functional group.

Particular preference is given to alkylsulfate and/or alkylether sulfate salts, (salts of) ether carboxylic acids, sarcosinates, isethionates, taurates, sulfosuccinates and/or alpha-olefin sulfonates, in particular alkylsulfate and/or alkylether sulfate salts.

Suitable amphoteric and/or zwitterionic surfactants within the meaning of the present invention are in particular mild surfactants having excellent foaming properties.

These are preferably understood to be surface-active compounds selected from the group comprising alkylbetaines, alkylamidoalkylbetaines, alkyl amphoacetates, alkyl amphodiacetates, alkyl amphopropionates, alkyl amphodipropionates, alkylsultaines, alkylhydroxysultaines, alkylamine oxides, alkyl amphoglycinates, alkyl iminodiacetates, alkyl iminodipropionates, alkyl amphopropyl sulfonates, alkyl amphocarboxyglycinates and alkyl amphocarboxypropionates.

Suitable alkylbetaines and/or alkylamidopropylbetaines preferably comprise C₄-C₂₄, more preferably C₆-C₁₈, particularly preferably C₈-C₁₄ alkyl chains which may be linear or branched, wherein linear is preferred. Particularly preferred alkylbetaines or alkylamidopropylbetaines are selected from the group of lauryl betaines, coco betaines, behenyl betaines, caprylic/capramidpropyl betaines, cetyl betaines, cocamidoethyl betaines, cocamidopropyl betaines, coco/oleamidopropyl betaines, decyl betaines, dimer dilinoleamidopropyl dibetaines, hydrogenated tallow betaines, hydroxylauryl/hydroxymyristyl betaines, isostearamidopropyl betaines, lauramidopropyl betaines, lauryl betaines, myristyl betaines, oleamidopropyl betaines, oleyl betaines, palmitamidopropyl betaines, ricinoleamidopropyl betaines, stearamidopropyl betaines, stearyl betaines, tallowamidopropyl betaines, tallow betaines, tallow dihydroxyethyl betaines, undecylenamidopropyl betaines, sunflower amidopropyl betaines, cetyl betaines, lauryl betaines.

Very particular preference is given to cocamidopropyl betaine.

Particularly suitable alkyl amphoacetates, alkyl amphodiacetates, alkyl amphopropionates or alkyl amphodipropionates are selected from the group of cocobetainamido amphopropionate, DEA-cocoamphodipropionate, disodium caproamphodiacetate, disodium caproamphodipropionate, disodium capryloamphodiacetate, disodium capryloamphodipropionate, disodium cocoamphodiacetate, disodium cocoamphodipropionate, disodium isostearoamphodiacetate, disodium isostearoamphodipropionate, disodium laureth-5 carboxyamphodiacetate, disodium lauroamphodiacetate, disodium lauroamphodipropionate, disodium oleoamphodipropionate, disodium PPG-2-isodeceth-7 carboxyamphodiacetate, disodium stearoamphodiacetate, disodium tallowamphodiacetate, sodium caproamphopropionate, sodium capryloamphoacetate, sodium capryloamphopropionate, sodium cocoamphoacetate, sodium cocoamphopropionate, sodium isostearoamphoacetate, sodium isostearoamphopropionate, sodium lauroamphoacetate, sodium lauroamphopropionate, sodium myristoamphoacetate, sodium oleoamphoacetate, sodium oleoamphopropionate, sodium stearoamphoacetate, sodium stearoamphopropionate, sodium tallamphopropionate, sodium tallowamphoacetate, sodium undecylenoamphoacetate and sodium undecylenoamphopropionate.

Very particular preference is given to sodium cocoamphoacetate and disodium cocoamphodiacetate.

Particularly suitable alkylsultaines or alkylhydroxysultaines are selected from the group of capryl sultaine, cocamidopropyl hydroxysultaine, coco-hydroxysultaine, coco-sultaine, erucamidopropyl hydroxysultaine, lauramidopropyl hydroxysultaine, lauryl hydroxysultaine, lauryl sultaine, myristamidopropyl hydroxysultaine, oleamidopropyl hydroxysultaine, tallowamidopropyl hydroxysultaine.

Particularly suitable alkylamine oxides are selected from the group of behenamine oxide, cocamidopropylamine oxide, cocamine oxide, decylamine oxide, decyltetradecylamine oxide, dihydroxyethyl C8-10 alkoxypropylamine oxide, dihydroxyethyl C9-11 alkoxypropylamine oxide, dihydroxyethyl C12-15 alkoxypropylamine oxide, dihydroxyethyl cocamine oxide, dihydroxyethyl lauramine oxide, dihydroxyethyl stearamine oxide, dihydroxyethyl tallowamine oxide, hydrogenated tallowamine oxide, isostearamidopropylamine oxide, lauramidopropylamine oxide, lauramine oxide, myristamidopropylamine oxide, myristamine oxide, palmitamidopropylamine oxide, palmitamine oxide, PEG-3 lauramine oxide, stearamidopropylamine oxide, stearamine oxide, tallowamidopropylamine oxide, tallowamine oxide, undecylenamidopropylamine oxide.

Particularly suitable alkyl amphoglycinates are selected from the group of caproamphoglycinate, capryloamphoglycinate, cocoamphoglycinate, isostearoamphoglycinate, lauroamphoglycinate, myristoamphoglycinate, oleoamphoglycinate, stearoamphoglycinate, tallowamphoglycinate, undecylenoamphoglycinate.

Particularly suitable alkyliminodiacetates or alkyliminodipropionates are selected from the group of disodium cocaminopropyl iminodiacetate, disodium hydroxyethyliminodiacetate, disodium lauriminodiacetate, disodium lauriminodipropionate, disodium steariminodipropionate, disodium tallowiminodipropionate, sodium C12-15 alkoxypropyl iminodipropionate, sodium cocoiminodiacetate, sodium lauriminodipropionate.

Particularly suitable alkyl amphopropyl sulfonates are selected from sodium cocoamphohydroxypropylsulfonate, sodium lauroamphohydroxypropylsulfonate, sodium oleoamphohydroxypropylsulfonate, sodium stearoamphohydroxypropylsulfonate, disodium
lauriminobishydroxypropylsulfonate, disodium cocoamphocarboxyethylhydroxypropylsuIfonate, sodium caproamphohydroxypropylsulfonate, sodium capryloamphohydroxypropylsulfonate.

Particularly preferably, antidandruff shampoos according to the invention contain alkylamidoalkylbetaines, alkyl amphoacetates and/or alkyl amphodiacetates.

Suitable non-ionic surfactants for use as a co-surfactant are, for example,
amine oxides, which can be selected from compounds of the general formulae (I) or (II)

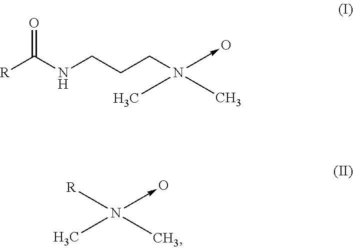

in which R in each case represents a straight-chain or branched, saturated or monounsaturated or polyunsaturated alkyl or alkenyl group having 6 to 24 carbon atoms, preferably having 8 to 18 carbon atoms.

The surfactants of the aforementioned formulae (I) or (II) that are known under the INCI names Cocamine Oxide, Lauramine Oxide, and/or Cocamidopropylamine Oxide and are commercially available from a number of suppliers are preferred in particular.
Fatty acid alkanolamides of the following general formula,

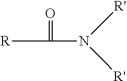

in which R preferably signifies a linear or branched saturated or unsaturated alkyl or alkenyl residue having 8 to 24 carbon atoms, and the residues R′ denote hydrogen or the group —(CH₂)ₙOH, in which n signifies the number 2 or 3, with the proviso that at least one of the residues R′ denotes the aforementioned residue —(CH₂)ₙOH,
mixtures of alkyl (oligo)glucosides and fatty alcohols, for example, the commercially available product Montanov®68,
sterols. Sterols are understood to refer to a group of steroids that bear a hydroxy group at the C atom 3 of the steroid structure, and are isolated both from animal tissue (zoosterols) and from vegetable fats (phytosterols). Examples of zoosterols include cholesterol and lanosterol. Examples of suitable phytosterols include ergosterol, stigmasterol, and sitosterol. There are also sterols that are isolated from fungi and yeasts (so-called mycosterols).
phospholipids. These are understood to mean principally the glucose phospholipids, which are obtained e.g., as lecithins or phosphatidylcholines from, for example, egg yolk or plant seeds (e.g., soybeans),
alkyl (oligo)glycosides. Suitable alkyl (oligo)glycosides can be selected from compounds of the general formula RO—[G]ₓ, in which [G] is preferably derived from aldoses and/or ketoses having 5-6 carbon atoms, preferably from glucose.

The index number x denotes the degree of oligomerization (DP), i.e. the distribution of mono- and oligoglycosides. The index number x preferably has a value in the range from 1 to 10, particularly preferably in the range from 1 to 3, wherein it need not be a whole number but can be a fraction which can be determined by analysis.

Particularly preferred alkyl (oligo)glycosides have a degree of oligomerization between 1.2 and 1.5. The residue R preferably denotes at least one alkyl and/or alkenyl residue having 4 to 24 C atoms.
Especially preferred alkyl (oligo)glycosides are compounds that are known under the INCI designations Caprylyl/Capryl Glucoside, Decyl Glucoside, Lauryl Glucoside, and Coco Glucoside.

Particularly preferably, antidandruff shampoos according to the invention contain fatty acid alkanolamides and/or alkyl (oligo)glucosides.

Alkyl (oligo)glucosides are particularly preferred.

In addition to the substantial and optional constituents mentioned above, the antidandruff shampoos according to the invention can contain other hair-conditioning active substances to enhance their care properties, as long as they do not adversely affect the foaming capacity and/or the stability of the antidandruff shampoos.

Particularly suitable hair-conditioning active substances within the meaning of the present invention are preferably
c) cationic polymers,
d) oil, fat and/or wax components,
e) protein hydrolyzates,
f) or mixtures thereof.

In a preferred embodiment, antidandruff shampoos according to the invention contain at least one cationic polymer c) in a proportion by weight of 0.01-3 wt. %, more preferably 0.02-2 wt. %, and very particularly preferably 0.025-1 wt. % based on the total weight of the antidandruff shampoo.

Suitable cationic polymers c) within the meaning of the present invention are, for example:
quaternized cellulose polymers, in particular Polyquaternium-10, such as those commercially available under the names Celquat® and Polymer JR®,
hydrophobically modified cationic cellulose derivatives, for example the cationic polymers distributed under the trade name SoftCat®,
cationic alkylpolyglycosides,
cationized honey, for example the commercial product Honeyquat® 50,
cationic galactomannan derivatives, in particular cationic guar and/or cassia polymers, such as the products available under the trade names Jaguar®, N-Hance®, Polycare®, Clearhance®. Jaguar® C-162, Jaguar® C500, Jaguar® Styl 100, Jaguar® Excel, N-Hance® 3196, N-Hance® HPCG 1000 and/or Polycare® Split Therapy are particularly suitable,
cationic starch derivatives, for example those commercially available under the name Mirustyle®,
cationic inulin polymers,
polymeric dimethyldiallylammonium salts and copolymers thereof with esters and amides of acrylic acid and methacrylic acid, in particular Polyquaternium-6 and Polyquaternium-7. The products commercially available under the names Merquat®100 (poly(dimethyldiallylammonium chloride)) and Merquat®550 (dimethyldiallylammonium chloride/acrylamide copolymer) are examples of such cationic polymers,
copolymers of vinylpyrrolidone with quaternized derivatives of dialkylaminoalkyl acrylate and methacrylate, such as vinylpyrrolidone-dimethylaminoethyl methacrylate copolymers quaternized with diethylsulfate. Such compounds are commercially available under the names Gafquat®734 and Gafquat®755,
vinylpyrrolidone-vinylimidazolium methochloride copolymers, such as offered under the names Luviquat® FC 370, FC 550, FC 905, and HM 552,
quaternized polyvinyl alcohol,
as well as the polymers known under the names
Polyquaternium-2, Polyquaternium-17, Polyquaternium-18, Polyquaternium-24, Polyquaternium-27, Polyquaternium-32, Polyquaternium-37, Polyquaternium-67, Polyquaternium-74 and Polyquaternium-89.

Preferred are cationic polymers of natural origin, such as quaternized cellulose polymers, hydrophobically modified cationic cellulose derivatives and/or cationic guar derivatives; particularly preferred are the cationic polymers known under the INCI names Guar Hydroxypropyltrimonium Chloride, Polyquaternium-10, Polyquaternium-6, Polyquaternium-37, Polyquaternium-67, or mixtures thereof.

In a further preferred embodiment, antidandruff shampoos according to the invention additionally contain 0.01-5 wt. %, more preferably 0.025-3 wt. %, and very particularly preferably 0.05-2.5 wt. % of at least one hair care substance d) different from c), wherein the quantities specified relate to the total weight of the antidandruff shampoo.

Suitable hair care substances e) within the meaning of the present invention are preferably understood to mean oil, fat and/or wax components that can be selected from, for example, glycerol esters, natural oils, butters or waxes, ester oils, ethers, C₈-C₁₆ alkanes, or mixtures thereof.

Suitable oil, fat and/or wax components within the meaning of the present invention are:
mineral oils such as paraffin and isoparaffin oils and synthetic hydrocarbons. Examples of hydrocarbons that can be used are, for example, the mixtures of alkanes available as commercial products (Cetiol® Ultimate) and/or 1,3-di-(2-ethylhexyl) cyclohexane (Cetiol® S).
dialkyl ethers, preferably di-n-alkyl ethers having in total between 12 and 36 C atoms, in particular 12 to 24 C atoms, such as for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether as well as di-tert-butyl ether, diisopentyl ether, di-3-ethyl decyl ether, tert-butyl n-octyl ether, isopentyl n-octyl ether and 2-methyl pentyl n-octyl ether. Particular preference is given to di-n-octyl ether, which is commercially available under the name Cetiol® OE.
saturated, mono- or polyunsaturated, branched or unbranched fatty alcohols having C₆-C₃₀, preferably C₁₀-C₂₂ and very particularly preferably C₁₂-C₂₂ carbon atoms can be used as fatty alcohols. Examples include decanol, octanol, octenol, dodecenol, decenol, octadienol, dodecadienol, decadienol, oleyl alcohol, erucic alcohol, ricinoleic alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol, and Guerbet alcohols thereof, this list being intended to be of exemplary and non-limiting nature. However, the fatty alcohols originate from preferably natural fatty acids, it usually being possible to start from an isolation from the esters of the fatty acids by reduction. According to the invention, use may also be made of those fatty alcohol fractions which are produced by reducing naturally occurring triglycerides such as beef tallow, palm oil, peanut oil, rapeseed oil, cottonseed oil, soybean oil, sunflower oil and linseed oil, or fatty acid esters which are produced from the transesterification products thereof with suitable alcohols, and thus represent a mixture of different fatty alcohols. Such substances are commercially available, for example, under the names Stenol®, e.g. Stenol® 1618 or Lanette®, e.g. Lanette® O or Lorol®, e.g. Lorol® C₈, Lorol® C₁₄, Lorol® C₁₈, Lorol® C₈-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, lanolin alcohols, such as those commercially available under the names Corona®, White Swan®, Coronet® or Fluilan®, can also be used according to the invention.
ester oils such as the esters of C₆-C₃₀ fatty acids with C₂-C₃₀ fatty alcohols. Preference is given to the monoesters of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof. Particularly preferred are isopropyl myristate (Rilanit® IPM), isononanoic acid C₁₆-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate, and diisotridecyl acelaate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
symmetrical, asymmetrical or cyclic esters of carbonic acid with fatty alcohols,
glycerol carbonate or dicaprylyl carbonate (Cetiol® CC),
ethoxylated or non-ethoxylated mono-, di- and trifatty acid esters of saturated and/or unsaturated linear and/or branched fatty acids with glycerol, for example, Monomuls® 90-018 (INCI name: Glyceryl Oleate), Monomuls® 90-L12 (INCI name: Glyceryl Laurate), Cetiol® HE (INCI name: PEG-7 Glyceryl Cocoate) or Cutina® MD (INCI name: Glyceryl Stearate).
natural oils and/or butters, such as coconut oil, (sweet) almond oil, walnut oil, peach kernel oil, apricot kernel oil, avocado oil, tea tree oil, soybean oil, cottonseed oil, sesame oil, sunflower oil, tsubaki oil, evening primrose oil, rice bran oil, palm oil, palm kernel oil, mango kernel oil, cranberry oil, sea buckthorn seed oil, meadow foam oil, thistle oil, macadamia nut oil, grapeseed oil, amaranth seed oil, argan oil, bamboo oil, olive oil, wheat germ oil, pumpkin seed oil, mallow oil, marula oil, pomegranate seed oil, paradise nut oil, passion fruit kernel oil, rose hip kernel oil, hazelnut oil, hemp oil, coffee oil, castor oil, safflower oil, corn oil, olive oil, rapeseed oil, canola oil, sasanqua oil, jojoba oil, rambutan oil, shea butter, mango butter, murumuru butter, cocoa butter, apricot kernel butter, mafura butter, bacuri butter, tucuma butter, ucuuba butter and/or cupuacu butter.
waxes, for example natural waxes such as carnauba wax, candelilla wax and/or jojoba oil, as well as animal waxes such as beeswax, wool wax, spermaceti and/or uropygial gland fat, and/or synthetic mineral waxes such as hard paraffin, ceresin, ozokerite, ester waxes such as polyethylene glycol or polyethylene glycol ester waxes and/or hydrogenated vegetable oils (in particular hydrogenated castor oil).

For some embodiments, it can be particularly advantageous if antidandruff shampoos according to the invention contain non-ethoxylated glycerol esters as care substance d).

In particular, glycerol mono- and/or diesters are suitable in this embodiment because they have a good oil-restoring capacity and nourish the scalp and hair without the occurrence of over-oiling. In addition, they do not impair the foaming properties of the antidandruff shampoos.

A glycerol ester particularly suitable in this context is glycerol monooleate.

In a further preferred embodiment, antidandruff shampoos contain at least one protein hydrolyzate in a proportion by weight of 0.01 to 3 wt. % based on the total weight of the antidandruff shampoo.

Suitable protein hydrolyzates are understood to mean product mixtures that can be obtained by acid-, base-, or enzyme-catalyzed degradation of proteins.

Protein hydrolyzates of plant, animal and/or marine origin can be used.

Animal protein hydrolysates are, for example, elastin, collagen, keratin, silk and milk egg white protein hydrolysates, which can also be in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolyzates of plant origin are preferred, for example soybean, almond, rice, pea, potato and wheat protein hydrolyzates. Such products are available, for example, under the trade names Gluadin® (Cognis), DiaMin® (Diamalt), Lexein® (Inolex) and Crotein® (Croda).

Cationized protein hydrolyzates can also be used, it being possible for the underlying protein hydrolyzate to originate from animals, for example from collagen, milk or keratin, from plants, for example from wheat, corn, rice, potatoes, soybeans or almonds, from marine life forms, for example from fish collagen or algae, or from biotechnologically obtained protein hydrolyzates. The protein hydrolyzates forming the basis of the cationic derivatives may be obtained from the corresponding proteins by chemical, in particular alkaline or acidic, hydrolysis, by enzymatic hydrolysis, and/or by a combination of both types of hydrolysis. The hydrolysis of proteins usually results in a protein hydrolyzate having a molecular weight distribution of approximately 100 daltons up to several thousand daltons. Cationic protein hydrolyzates of which the underlying protein portion has a molecular weight of from 100 to 25,000 daltons, preferably from 250 to 5,000 daltons, are preferred. Furthermore, cationic protein hydrolyzates are understood to mean quaternized amino acids and mixtures thereof. Quaternization of the protein hydrolyzates or of the amino acids is often carried out by utilizing quaternary ammonium salts such as, for example, N,N-dimethyl-N-(n-alkyl)-N-(2-hydroxy-3-chloro-n-propyl)ammonium halides. Furthermore, the cationic protein hydrolyzates can also be derivatized even further. Mentioned as typical examples of the cationic protein hydrolyzates and derivatives are the products that are commercially available and are known under the INCI names: cocodimonium hydroxypropyl hydrolyzed collagen, cocodimopnium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxyproypltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryldimonium hydroxypropyl hydrolyzed casein, lauryldimonium hydroxypropyl hydrolyzed collagen, lauryldimonium hydroxypropyl hydrolyzed keratin, lauryldimonium hydroxypropyl hydrolyzed silk, lauryldimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein.

Other active substances, auxiliary substances and additives that can preferably be contained in the antidandruff shampoos according to the invention are, for example:
perfume,
UV filters,
(natural) dyes for coloring the agent,
active ingredients such as bisabolol and/or allantoin,
complexing agents such as EDTA, NTA, β-alanine diacetic acid, and phosphonic acids,
ceramides. Ceramides are understood to mean N-acylsphingosine (fatty acid amides of sphingosine) or synthetic analogs of such lipids (so-called pseudo-ceramides),
antioxidants,
preservatives, such as for example sodium benzoate or salicylic acid,
pH setting agents such as sodium hydroxide, citric acid, and/or lactic acid,
viscosity regulators such as salts (NaCl).

A second subject matter of the invention is the cosmetic use of the antidandruff shampoo according to the invention for cleaning and caring for human hair and for the prophylaxis, reduction, elimination and alleviation of dandruff on hair-covered body surfaces.

A third subject matter of the invention is a cosmetic method for controlling scalp dandruff in which an antidandruff shampoo containing
a) at least one compound according to formula (I) and
b) at least one surfactant
is applied to the hair and scalp and rinsed out after a contact time of 5 seconds to 5 minutes.

The statements made about the compositions according to the invention apply in regard to other preferred embodiments of the use according to the invention and the method according to the invention.

The following examples are intended to explain the subject matter of the present invention in a non-limiting manner.

### Examples

The following antidandruff shampoos were prepared: The quantities specified in the tables are percentages by weight.

| | 1 | 2 | 3 |
|---|---|---|---|
| Compound according to formula (I) | 0.1-3 | | |
| Methylheptylglycerin | | 0.2-2 | 0.3-1.5 |
| Surfactant | 0.5-20 | | |
| Anionic surfactant | | 1-15 | 2.5-15 |
| Amphoteric or zwitterionic surfactant | | 0-7.5 | 0.1-5 |
| Non-ionic surfactant | | 0-5 | 0-3 |
| Water and optional further auxiliaries | up to 100 | up to 100 | up to 100 |
| and active ingredients | | | |
| | 4 | 5 | 6 |
| Methylheptylglycerin | 0.1-3 | 0.1-3 | 0.1-3 |
| Anionic surfactant | 1-15 | 1-15 | 1-15 |
| Amphoteric or zwitterionic surfactant | 0-7.5 | 0-7.5 | 0-7.5 |
| Non-ionic surfactant | 0-5 | 0-5 | 0-5 |
| Cationic polymer c) | 0-3.5 | 0-3.5 | 0-3.5 |
| Caring substance d) | 0-5 | 0-5 | 0-5 |
| Water and optional further auxiliaries | up to 100 | up to 100 | up to 100 |
| and active ingredients | | | |

| | 7 | 8 | 9 | 10 |
|---|---|---|---|---|
| Methylheptylglycerin | 0.1-3 | 0.2-2 | 0.3-1.5 | 0.3-1 |
| Sodium laureth sulfate | 1-15 | 1-15 | 1-15 | 7-10 |
| Cocamidopropyl betaine | 0-7.5 | 0.1-6 | 0.5-5 | 1-2 |
| Lamesoft ® PO 65* | 0-5 | 0-5 | 0.5-5 | 1-2 |
| Guar | 0-3.5 | 0.01-1 | 0.025-0.5 | 0.01-0.5 |
| hydroxypropyltrimonium | | | | |
| chloride | | | | |
| Water and optional | up to | up to | up to | up to |
| further auxiliaries | 100 | 100 | 100 | 100 |
| and active ingredients | | | | |

*INCI name: Coco Glucoside and Glyceryl Oleate (approx. 65% AS); BASF

The antidandruff shampoos according to the invention allow effective and sustainable treatment of dandruff conditions. In particular on sensitive scalps, they additionally have a good and mild cleaning, balancing and soothing effect.

What is claimed is:

1. An antidandruff shampoo, including a cosmetic carrier, comprising
a) 0.05-5 wt. % of at least one compound of formula (I)
R¹—O—[CH₂—CH(OH)—CH₂—O]ₙ—H (I)
where
R¹ represents a branched C₆-C₁₆ alkyl, alkenyl or alkynyl group
n represents a number of 1-3, and
b) 0.5-20 wt. % of at least one surfactant,
c) 0.01-3 wt. % of at least one cationic polymer,
d) 0.01-5 wt. % of at least one non-ethoxylated glycerol ester hair care substance d) different from c)
wherein the quantities specified relate to the total weight of the antidandruff shampoo and
wherein the at least one compound according to formula (I) includes H₃C—(CH₂)₅—CH(CH₃)—O—CH₂—CH(OH)—CH₂OH (methylheptylglycerin).

2. The antidandruff shampoo according to claim 1, wherein in the at least one compound of formula (I) n represents the number 1 and R¹ represents a branched C₆-C₁₂ alkyl, alkenyl or alkynyl group.

3. The antidandruff shampoo according to claim 1, wherein the at least one surfactant b) is selected from the group consisting of: anionic surfactants, amphoteric surfactants, zwitterionic surfactants, non-ionic surfactants, and mixtures thereof.

4. The antidandruff shampoo according to claim 1, wherein the at least one surfactant b) comprises:
b1) 1-15 wt. % of at least one anionic surfactant,
b2) 0-7.5 wt. % of at least one amphoteric or zwitterionic surfactant,
b3) 0-5 wt. % of at least one non-ionic surfactant,
wherein the quantities specified relate to the total weight of the antidandruff shampoo.

5. The antidandruff shampoo according to claim 1, further comprising 0.01-3 wt. % of at least one cationic polymer c), based on the total weight of the antidandruff shampoo.

6. The antidandruff shampoo according to claim 1, wherein the at least one cationic polymer c) is selected from the group consisting of: Guar Hydroxypropyltrimonium Chloride, Polyquaternium-10, Polyquaternium-6, Polyquaternium-67, Polyquaternium-37, and mixtures thereof.

7. The antidandruff shampoo according to claim 1, further comprising 0.01-5 wt. % of at least one hair care substance d), said at least one hair care substance d) being different from the at least one cationic polymer c), wherein the quantities specified relate to the total weight of the antidandruff shampoo.

8. The antidandruff shampoo according to claim 7, wherein the at least one hair care substance is from glycerol monooleate.

9. A cosmetic method for controlling scalp dandruff, comprising applying the antidandruff shampoo according to claim 1 to hair or scalp and rinsing the hair or scalp after a contact time of 5 seconds to 5 minutes.
